(19) United States
(12) Patent Application Publication
(10) Pub. No.: US 2026/0001094 A1
(43) Pub. Date: Jan. 1, 2026

(54) APPARATUS FOR GENERATING DRY MIST

(71) Applicants: Bryan K. Wilcox, Cedar City, UT (US); Kenton T. Meadows, Madison, MA (US)

(72) Inventors: Bryan K. Wilcox, Cedar City, UT (US); Kenton T. Meadows, Madison, MA (US)

(21) Appl. No.: 19/322,115

(22) Filed: Sep. 8, 2025

Related U.S. Application Data
(63) Continuation-in-part of application No. 17/932,581, filed on Sep. 15, 2022, now Pat. No. 12,409,469.
(60) Provisional application No. 63/244,544, filed on Sep. 15, 2021.
(60) Provisional application No. 63/775,786, filed on Mar. 21, 2025.

(51) Int. Cl. B05B 17/06; A61L 2/22; A61L 2/24; A61L 9/14; A61L 101/06; A61L 101/20; B05B 7/00

(52) U.S. Cl. CPC B05B 17/0615; A61L 2/22; A61L 2/24; A61L 9/14; B05B 7/0012; B05B 7/0093; A61L 2101/06; A61L 2101/20; A61L 2202/11; A61L 2202/121; A61L 2202/14; A61L 2202/15; A61L 2202/16; A61L 2202/25; A61L 2209/111; A61L 2209/132; A61L 2209/133; A61L 2209/134

(57) ABSTRACT

A dry mist generating apparatus has a housing, an ultrasonic transducer, a fan inducing airflow within the plastic housing, a diffuser plate for directing airflow, increasing velocity, and decreasing pressure, at least one opening for releasing the resulting dry mist, and a reservoir for capturing droplets that exceed 4 microns. A dry mist system may include one or more sensors, communication devices, and processors, which are all configured to monitor and automatically sanitize a given area in response to a triggering event, such as detecting airborne pathogens. Separate zones may be defined, with each having at least one sensor and/or a dry mist generating apparatus. Some dry mist systems may be sized so as to be portable and placeable on a desk or counter with a swappable reservoir.

### CROSS-REFERENCE TO RELATED APPLICATIONS

This application is a continuation-in-part of U.S. Non-Provisional application Ser. No. 17/932,581, filed Sep. 15, 2022, which claimed the benefit of U.S. Provisional App. Ser. No. 63/244,544, filed on Sep. 15, 2021, and this application further claims the benefit of U.S. Provisional App. Ser. No. 63/775,786, filed on Mar. 21, 2025, all of which are incorporated herein by reference.

### TECHNICAL FIELD

The present disclosure relates to airborne mists. More particularly, the present disclosure relates to an apparatus for generating a dry mist.

### BACKGROUND

Pathogens, such as bacteria, viruses, mold, and mildew may be easily spread by remaining on surfaces or by passing through the air. Disinfectants have been used to reduce the spread of these pathogens, but their effectiveness is limited. For example, most disinfectants are of a liquid solution and must be either wiped or sprayed onto a surface. Spraying disinfectant into the air, via an applicator, is not effective as the droplets fall to the ground and surface much too fast due to the droplet size and weight. As a result, current disinfectants are inadequate for neutralizing airborne pathogens. Additionally, disinfectant sprays and solutions leave surfaces wet, which is not ideal and may not be safe for many surfaces, such as those with electronics. Disinfectant sprays and sprayers on the market typically do not achieve a micron size smaller than 10. As a result, the droplets are subject to gravity and will burst when landing on a surface, creating a wet spot. As a result, users must typically wipe the surfaces after using these sprayers.

Liquid droplets for cleaning may be separated into 3 major groups:
First, there are the 50 micron and larger droplets that are produced from hand-pump sprayers. Due to their size and mass, gravity pulls them down to the surface. This liquid must then be wiped, spread, or dried after the application. Typical spray glass and bathroom cleaners are examples.
Second, there are 10-50 micron droplets that come from high pressure sprayers with ultra-small orifices in the nozzle. Utilizing hundreds of pounds per square inch pressure, the liquid is forced through these small orifices to break the liquid into small droplets. While these are much smaller than the above-mentioned droplets, they also are subject to gravity and will be pulled down to surfaces and the droplet will “burst” on impact and become a wet spot. As with the above, drying, wiping, and spreading the liquid will be required.
Third, there are 1-9 micron droplets (referred to as dry mist) that are produced by ultrasonic transducers vibrating thousands of times per second. The ultrasonic transducer produces water droplets in all sizes from 1-200 microns. However, 1-9 micron size droplets are not influenced by gravity and remain suspended for hours and/or days based on heat and humidity.

Accordingly, there is a need for a dry mist (e.g., 1-9 micron droplets) disinfectant. In other words, there is a need for a disinfectant that can remain airborne for a significant amount of time, that may penetrate small spaces, and that does not leave surfaces wet. Additionally, there is a need for an apparatus that may produce this dry mist that is not susceptible to corrosion and other component failures. Current dry mist chambers and foggers are designed using metal components, which will not withstand prolonged exposure to hypochlorous acid (HOCL) and water. Delivering HOCL as a dry-mist disinfectant fog with traditional methods results in rapid failure of the metal parts in sprayers, nozzles, chambers, and pumps. Further, the mist expelled needs to be 9 microns and smaller, and ideally less than 4 microns, which has not been consistently achieved using the prior art. While examples are used above that demonstrate the need for dry mist disinfectants, it will be appreciated that the invention is not limited to disinfectant applications. For example, non-disinfectant dry mist (e.g., water) is also needed in plant growth, humidification, and other industries. Accordingly, the present disclosure seeks to solve these and other problems.

### SUMMARY OF EXAMPLE EMBODIMENTS

In some embodiments, a dry mist generating apparatus comprises a plastic housing, an ultrasonic transducer, a fan inducing air within the plastic housing, a diffuser plate for controlling direction, velocity, and pressure of the induced air, at least one discharge opening for releasing the resulting dry mist, and a reservoir for capturing droplets that exceed 4 microns. In some embodiments, the ultrasonic transducer is an ultrasonic disc.

In some embodiments, a dry mist generating apparatus further comprises one or more discharge tubes for releasing the dry mist. The length of the tubes may be varied, along with their diameter and angle, to achieve varying micron droplet sizes at discharge.

In some embodiments, a dry mist generating apparatus comprises one or more wheels for easy transportation. In one embodiment, a dry mist generating apparatus may be placed in a cart or carriage.

In some embodiments, a dry mist generating apparatus may be network-connected and configured to interface with one or more sensors, communication devices, controllers, or other components.

In some embodiments, a dry mist system comprises an enclosure, which may be sized so as to be portable, and may include a swappable reservoir.

### BRIEF DESCRIPTION OF THE DRAWINGS

FIG. 1 illustrates a top, front, left perspective view of a dry mist generating apparatus;

FIG. 2 illustrates a top, front, left perspective view of a dry mist generating apparatus with a transparent housing for ease of viewing internal components;

FIG. 3 illustrates a right side elevation view;

FIG. 4 illustrates a front elevation view of a dry mist generating apparatus;

FIG. 5 illustrates a left side elevation view of a dry mist generating apparatus;

FIG. 6 illustrates a rear elevation view of a dry mist generating apparatus;

FIG. 7 illustrates a top plan view of a dry mist generating apparatus;

FIG. 8 illustrates a front, right side perspective view with arrows illustrating airflow of a dry mist generating apparatus;

FIG. 9 illustrates a right elevation view with arrows illustrating airflow of a dry mist generating apparatus;

FIG. 10 illustrates a top plan view of a dry mist generating apparatus;

FIG. 11 illustrates a top, front, left side perspective view of a dry mist generating apparatus;

FIG. 12 illustrates a top, left side perspective view of a dry mist generating apparatus;

FIG. 13 illustrates a top, front, left side perspective view of a dry mist generating apparatus;

FIG. 14 illustrates a top, front, left side perspective view of a dry mist generating apparatus with the sidewalls of a cart removed for ease of viewing interior components;

FIG. 15 illustrates a left side elevation view of a dry mist generating apparatus;

FIG. 16 illustrates a longitudinal cross-section of a dry mist generating apparatus;

FIG. 17 illustrates a top, rear perspective view of a dry mist generating apparatus;

FIG. 18 illustrates a top, front, right side perspective view of a dry mist generating apparatus;

FIG. 19 illustrates a front, top perspective view of a dry mist generating apparatus;

FIG. 20 illustrates a detailed, top perspective view of a dry mist generating apparatus with a lid removed;

FIG. 21 illustrates a detailed perspective view of pipes, valves, and sensors of a dry mist generating apparatus;

FIG. 22 illustrates a detailed perspective view of pipes, valves, and sensors of a dry mist generating apparatus;

FIG. 23 illustrates a rear detailed perspective view of a dry mist generating apparatus with a lid in an open position;

FIG. 24 illustrates a top, right side, front perspective view of a dry mist generating apparatus;

FIG. 25 illustrates a top, right side, front perspective view of a dry mist generating apparatus;

FIG. 26 illustrates a diagram of a network-connected dry mist generating apparatus;

FIG. 27 illustrates a diagram of a network-connected dry mist generating apparatus;

FIG. 28 illustrates a top, side perspective view of a dry mist generating apparatus;

FIG. 29 illustrates a top, side perspective view of a dry mist generating apparatus, wherein the housing is transparent;

FIG. 30 illustrates a top perspective view of a dry mist generating apparatus with the top removed;

FIG. 31 illustrates a top, side perspective view of a dry mist generating apparatus with the top removed;

FIG. 32 illustrates a top plan view of a dry mist generating apparatus with the top removed;

FIG. 33 illustrates a front perspective view of a flow restricting plate of a dry mist generating apparatus;

FIG. 34 illustrates a front plan view of a flow restricting plate of a dry mist generating apparatus;

FIG. 35 illustrates a top, side perspective view of a dry mist generating apparatus and within a wheeled cart;

FIG. 36 illustrates a front, right perspective view of a dry mist system;

FIG. 37 illustrates a rear, left perspective view of a dry mist system; and

FIG. 38 illustrates a front, right, cross-sectional view of a dry mist system.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for a mist that can remain airborne for a significant amount of time, that may penetrate small spaces, and that does not leave surfaces wet, which may be useful in a variety of industries, including, but not limited to, disinfecting, humidifying, plant growth, etc. Additionally, there is a need for an apparatus that may produce this dry mist that is not susceptible to corrosion and other component failures. The apparatus for generating dry mist disclosed herein solves these and other problems.

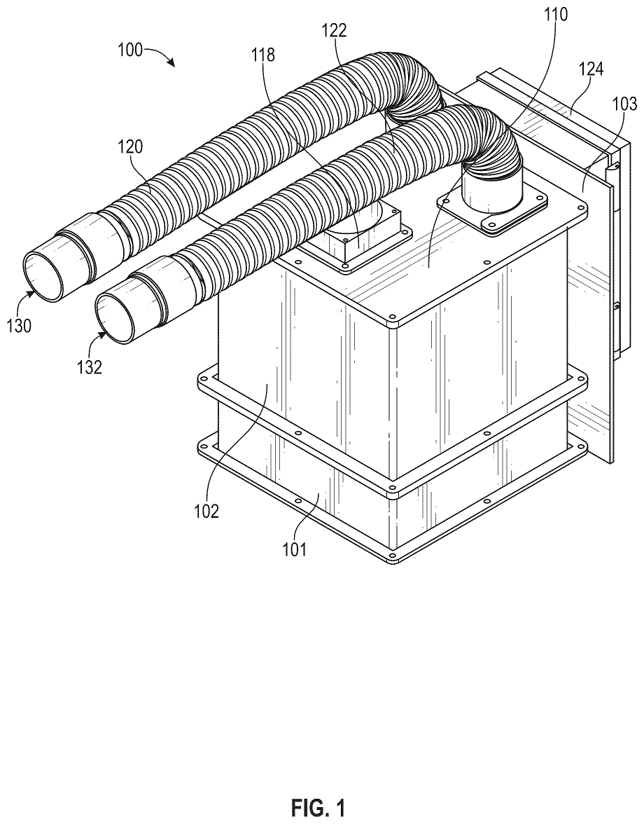

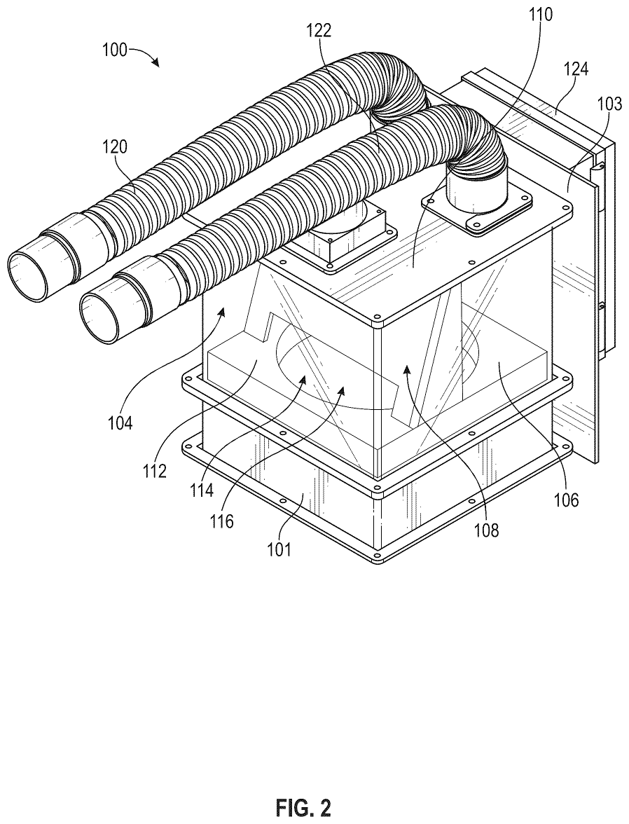

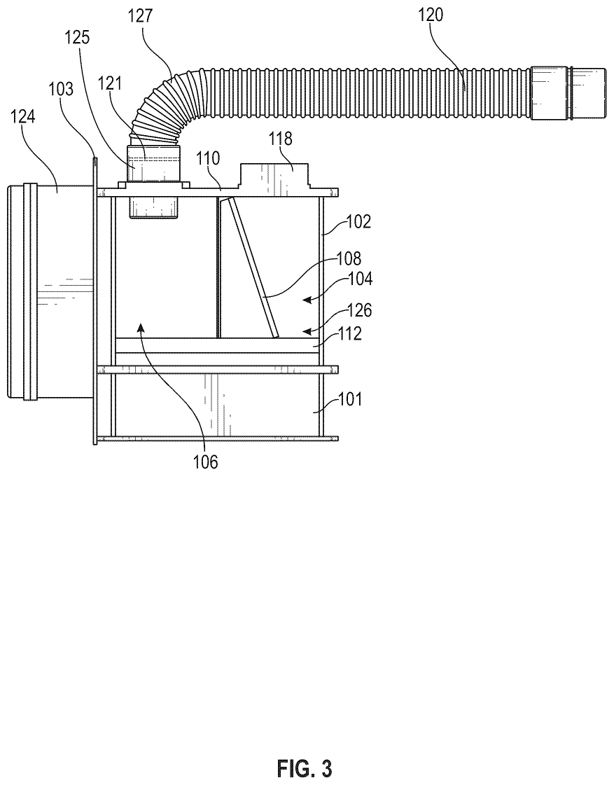

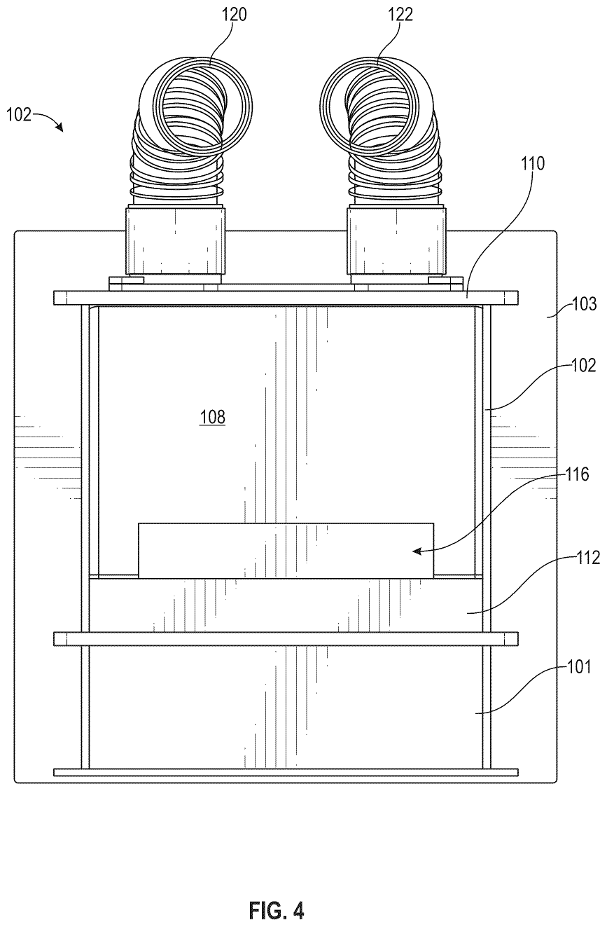

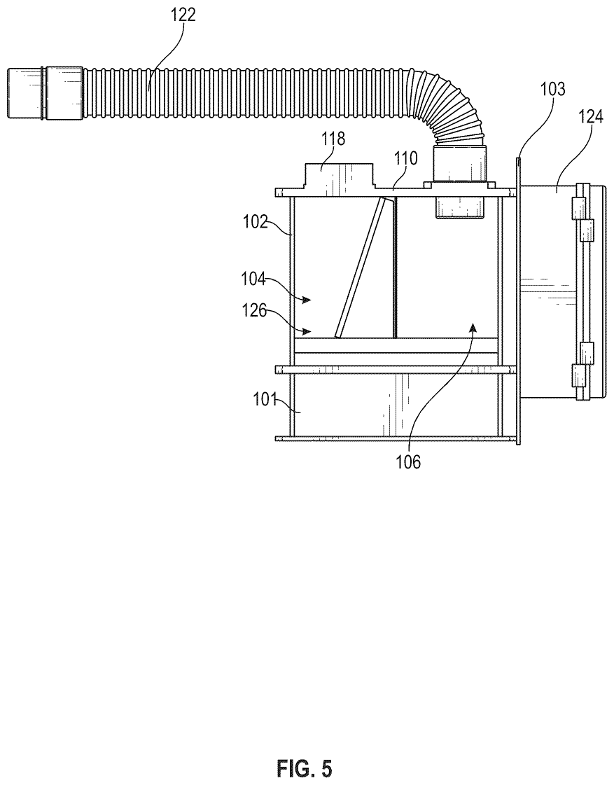

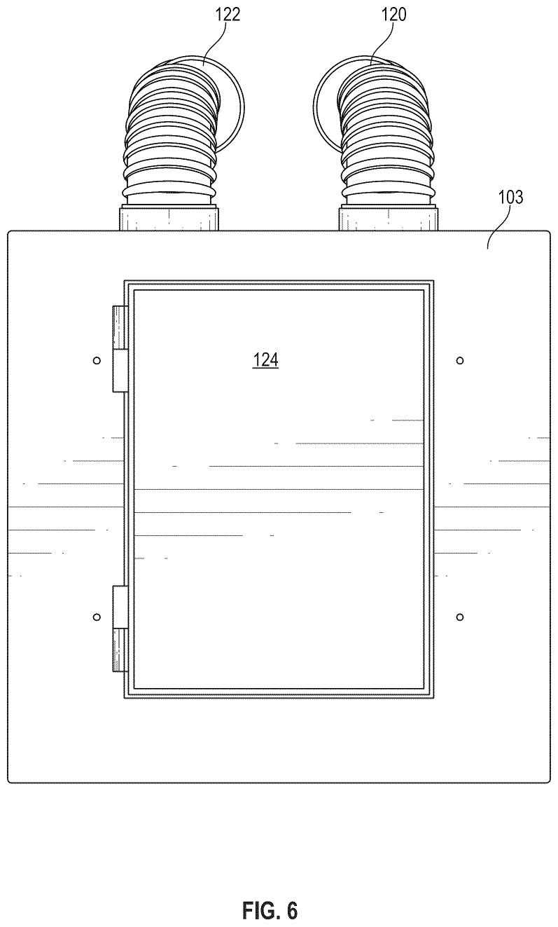

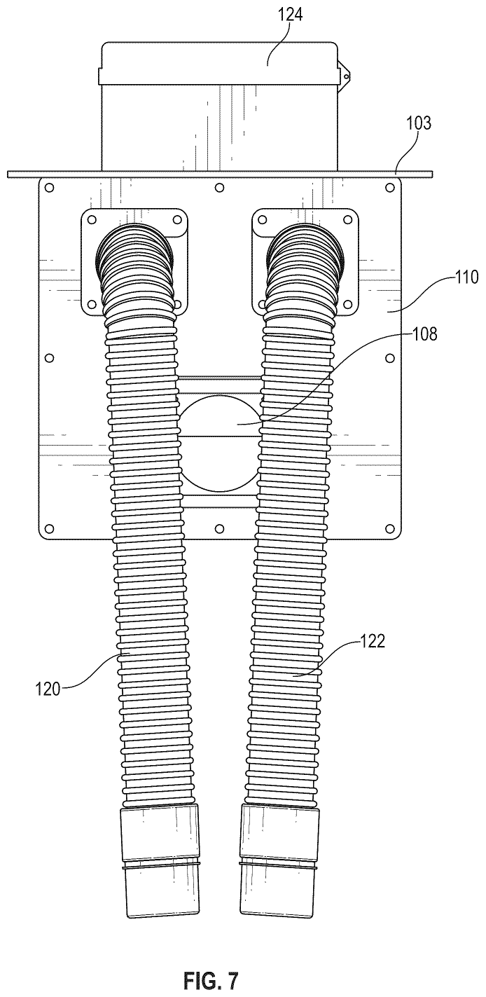

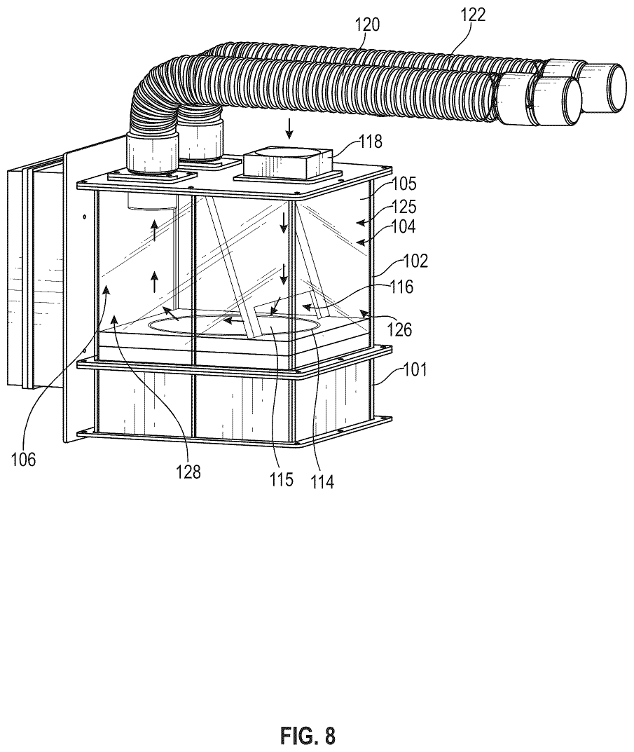

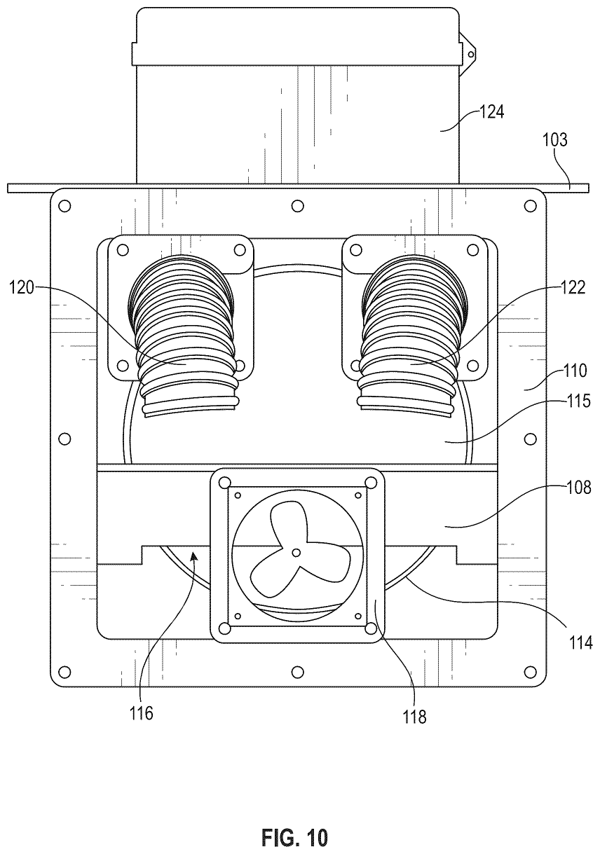

In some embodiments, as shown in FIGS. 1-10, a dry mist generating apparatus 100 comprises a housing 102 (which may be made from plastic, glass, stainless steel, or other materials that are resistant to corrosion) having a first chamber 104 and a second chamber 106 separated by a diffuser plate 108. As shown, the diffuser plate 108 is non-perpendicular (e.g., angled 45 degrees) to a top 110 and a base 112 of housing 102. The base 112 comprises an ultrasonic transducer (e.g., ultrasonic disc) aperture 114 configured to receive an ultrasonic transducer 115 (best seen in FIGS. 8 & 10). The diffuser plate 108 comprises an air aperture 116 near the base 112 and straddling the ultrasonic transducer aperture 114 that allows for the passage of air and mist from the first chamber 104 to the second chamber 106. A fan 118 forces air into the top of the first chamber 104 where the diffuser plate 108 directs the air to the lower, front of the first chamber 104 (i.e., a narrow neck 126, FIG. 3). The narrow neck 126 increases the velocity of the air and reduces pressure. The air then passes above the ultrasonic transducer via the air aperture 116 of the diffuser plate 108 and into the second chamber 106. Because of the reduced air pressure when entering the second chamber 106, the air lifts the dry mist (ideally about 3 microns or less, but range from 1-9 microns), which is created by an ultrasonic transducer within ultrasonic transducer aperture 114, to force it out through one or more discharge tubes 120, 122.

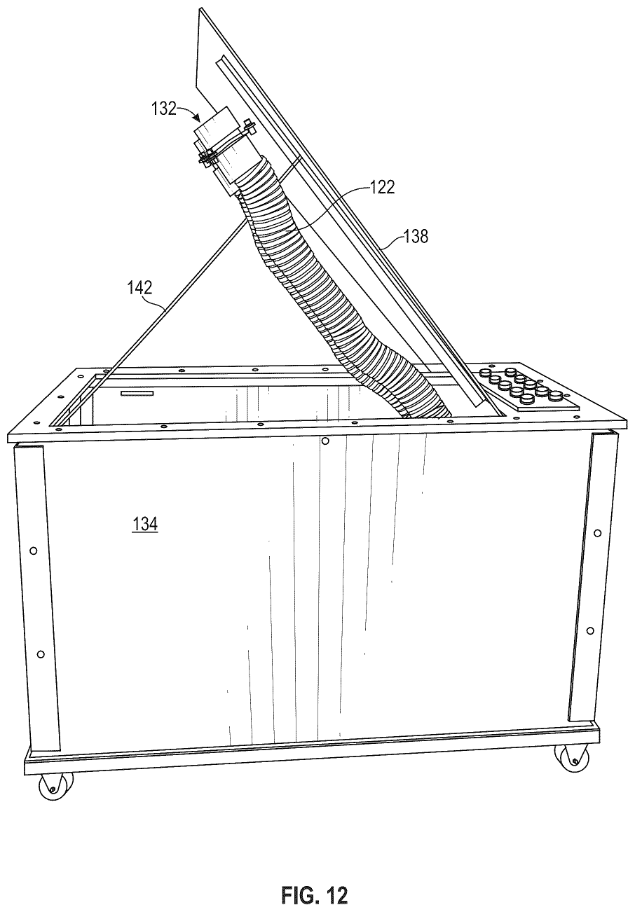

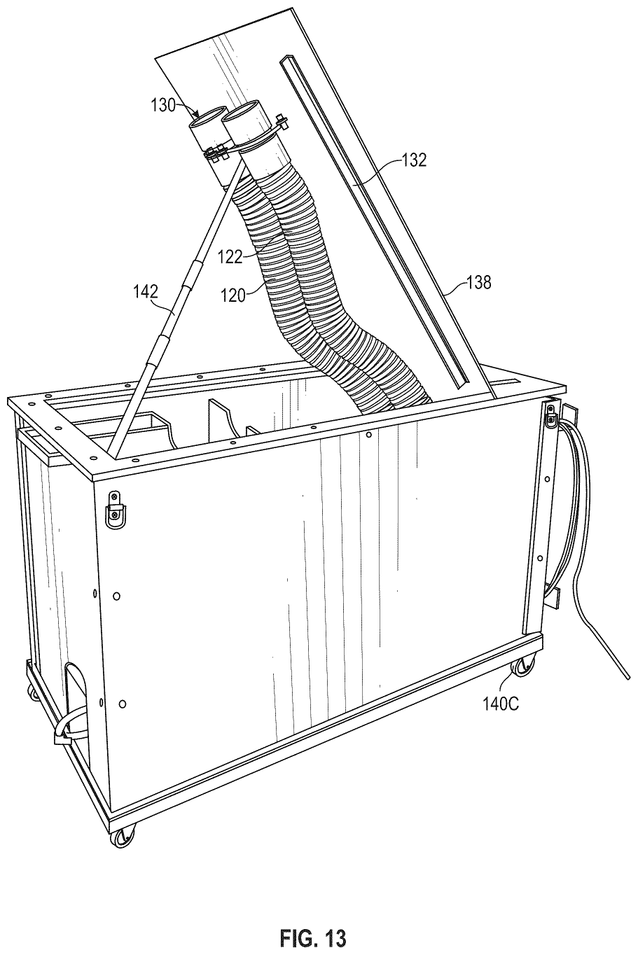

Droplets that exceed 4 microns remain in the reservoir (bottom of chambers 104, 106) by condensing and falling back to the chambers 104, 106. For example, liquid is added to the housing 102 where it is then converted to a dry mist via an ultrasonic transducer located at the base of the housing 102. The fan 118 then causes air to flow from the first chamber 104, under the diffuser plate 108, and into the second chamber 106 where it then rises upwardly to exit through the discharge tubes 120, 122 at the top of the housing 102. As the air travels upward to the discharge tubes 120, 122, the dry mist is carried in the air upward and outward through the discharge tubes 120, 122 as well. If any droplets larger than 4 microns happen to enter the discharge tubes 120, 122, the droplets condense on the sides of the tubes and fall back into the second chamber 106. To ensure this occurs, the discharge tubes 120, 122 are, ideally, non-linear. In other words, the discharge tubes 120, 122 have bends that aid in collecting droplets excess in size, as best shown in FIGS. 12-13. This non-linear configuration is most readily achieved using flexible tubing. However, it will be appreciated that non-flexible tubing that is shaped to be non-linear may also be used.

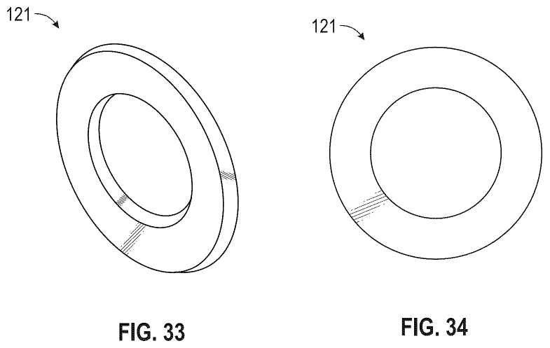

Additionally, each discharge tube 120, 122 may comprise a respective flow restricting plate 121 (FIGS. 33-34) therein. For example, as best shown in FIG. 3, the flow restricting plate 121 may be positioned within the cuff 123 of the discharge tube 120. The flow restricting plate 121 causes a pressure drop inside the discharge tube 120. As a result, with the pressure drop and bend 127 in the discharge tube 120, larger, heavier particles (e.g., particles larger than 2 microns) fall and return back to the second chamber 106 as liquid. This helps to ensure that particles of 2 microns or less exit the discharge tube 120. Because the fan speed is not reduced, the air continues to flow through the chambers 104, 106 at the desired rate (e.g., 120 cfm). While 120 cfm was provided as an example, it will be appreciated that other rates, both lower and higher, may be used without departing herefrom.

The dry mist generating apparatus 100 further comprises a controller 124 (e.g., microcontroller) which may be coupled to the housing 102 via a mounting plate 103. The controller 124 is configured to control the power status of the dry mist generating apparatus 100, as well as monitor various components of the dry mist generating apparatus 100, as will be discussed in greater detail later herein.

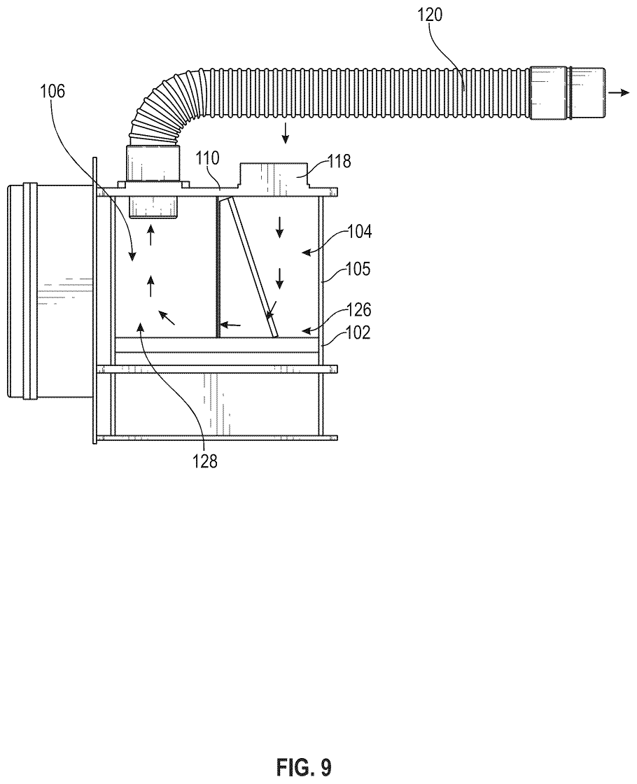

As shown in FIGS. 8-9, circular rotation air flow, which is turbulent and vortex, is induced into the first chamber 104 of the housing 102 using the fan 118. The air pathway is shown using arrows. Due to the angle of the diffuser plate 108, a narrow neck 126 is formed at the bottom of the first chamber 104. In other words, a top portion 125 of the first chamber 104 has a first distance from the diffuser plate 108 to a front wall 105 of the housing 102. The diffuser plate 108 is angled so that the lower end is closer to the front wall 105, forming the narrow neck 126. Because the turbulent and vortex air is compressed into the narrow neck 126 using the diffuser plate 108, the velocity of the air is increased, thereby increasing the pressure. The air then passes through the air aperture 116 of the diffuser plate 108 and above the ultrasonic transducer 115. As the air enters the second chamber 106, the larger volume space allows the air pressure to drop (e.g., Venturi effect) and the turbulent and vortex air flow from the first chamber 104 is flattened into straight laminar airflow over the ultrasonic transducer 115. The ultrasonic transducer 115 creates droplets, with some less than 10 microns in size. As the air passes into a bottom portion 128 of the second chamber 106, airflow is slowed, decreasing (e.g., Venturi effect). pressure and allowing accumulation. The heavier droplets fall while the smaller, sub-10 micron sized droplets proceed to the discharge tubes 120, 122. In some embodiments, it is preferable to produce droplets smaller than 5 microns so that they may pass through an HVAC unit, continuing to disinfect.

The length of the discharge tubes 120, 122, along with their diameter and bend, may be varied to achieve varying micron-sized droplets at discharge to the atmosphere. For example, the longer the discharge tubes 120, 122, the smaller the micron-sized droplets that exit. Conversely, shorter discharge tubes 120, 122 allow for larger droplets to be expelled. Additionally, the size of the ultrasonic transducer may also be used to control the size of the droplets. Accordingly, a user may adjust the discharge tubes 120, 122 and their bends in order to achieve the maximum desired micron size of the droplets at an exit 130, 132 of each discharge tube 120, 122. In order to achieve micron sizes less than 4 microns, the discharge tubes 120, 122 are ideally non-linear, comprising bends as shown in FIGS. 12-13. By achieving droplets that have a size of less than 4 microns, the droplets remain suspended in the air and may freely flow therewith (defined herein as a “dry mist”). This dry mist is then able to penetrate all areas of a space or building as it flows through the air, thereby disinfecting the space or building as it penetrates.

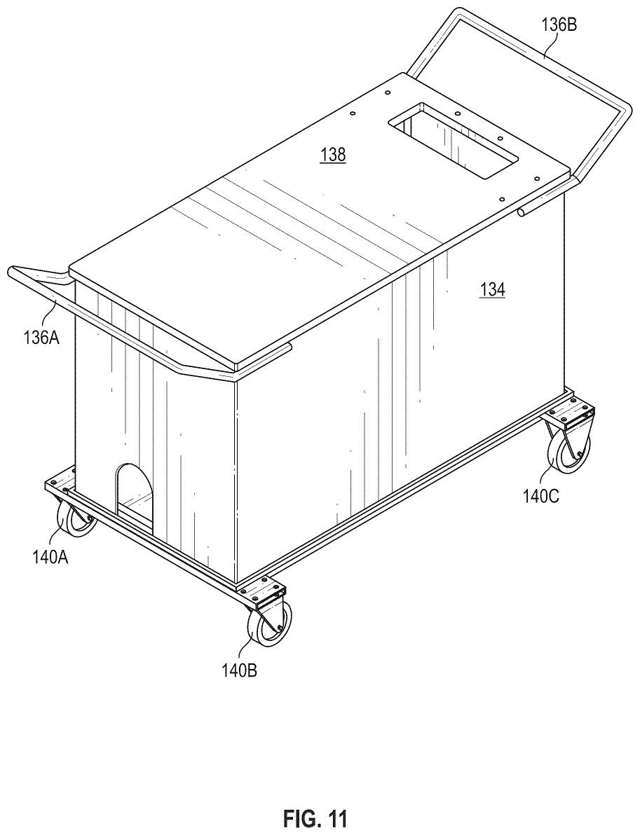

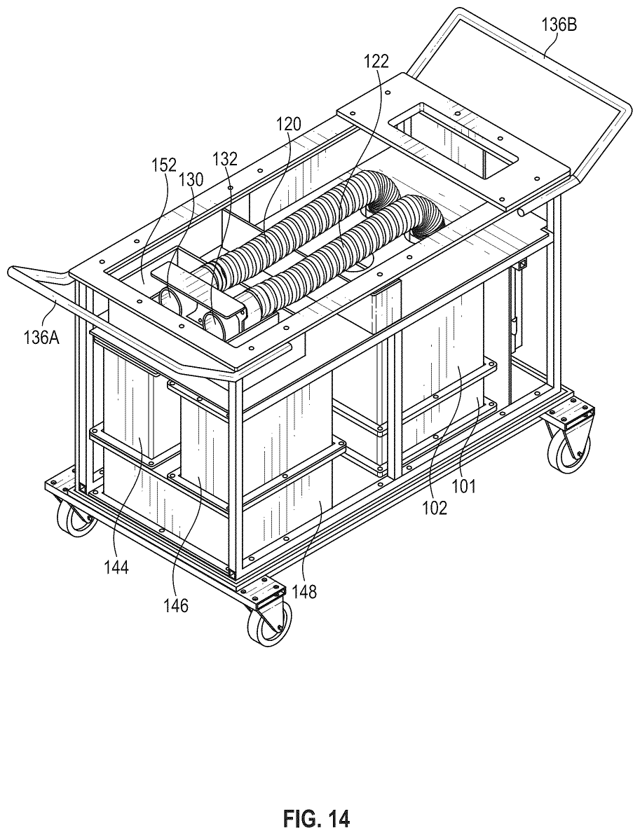

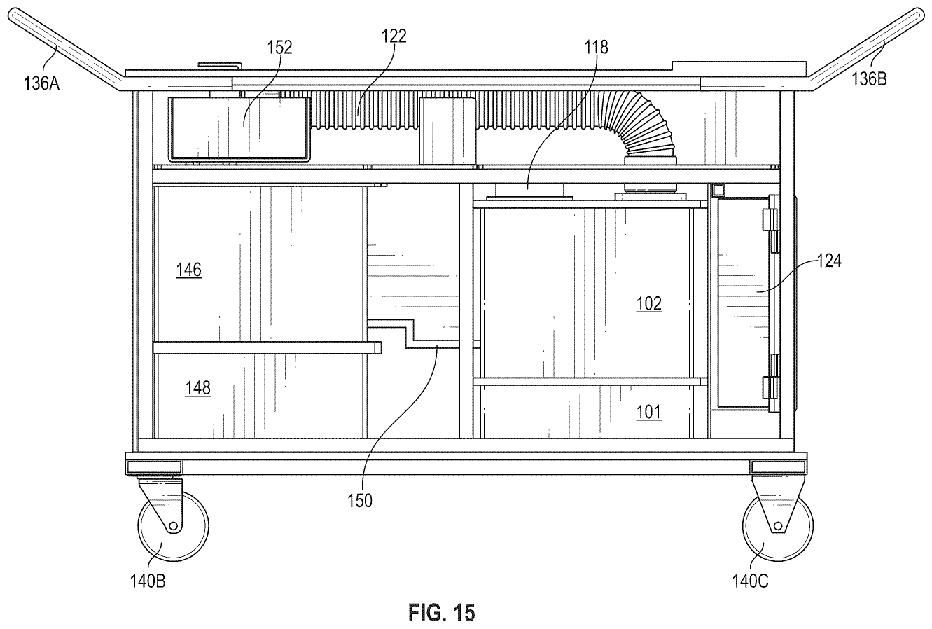

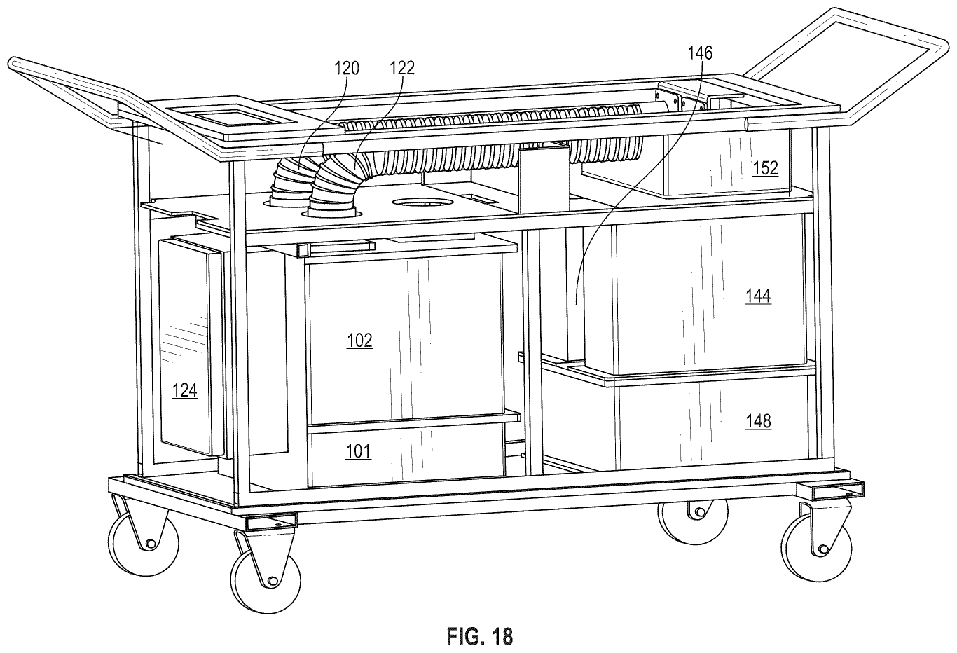

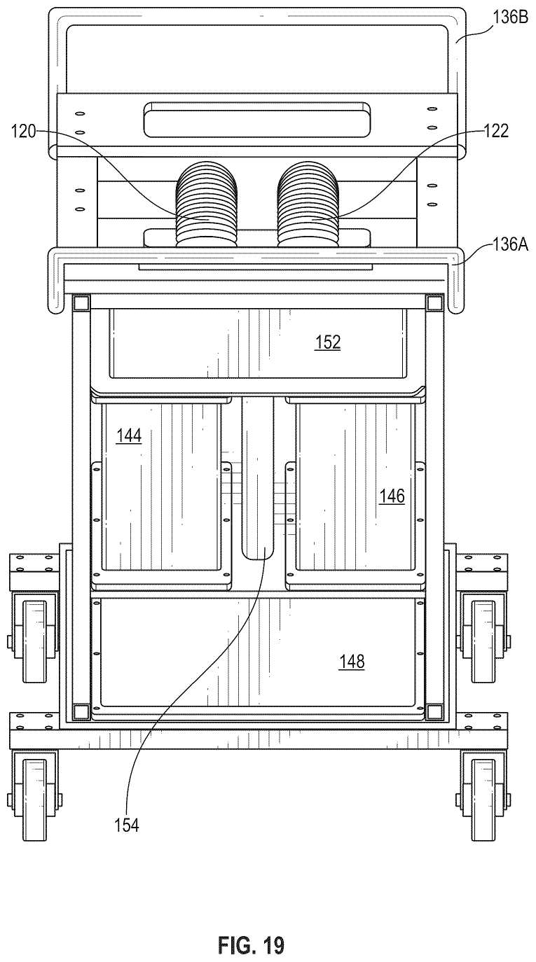

In some embodiments, as shown in FIGS. 11-23, the dry mist generating apparatus 100 may be contained within a wheeled cart 134, the wheeled cart 134 comprising one or more handles 136A-B, a hinged lid 138, and a plurality of wheels 140A-D. As shown in FIGS. 12-13, the hinged lid 138 can secured in an open position, such as by using a rod 142, with the discharge tubes 120, 122 coupled to the hinged lid 138 such that they are raised with the hinged lid 138. As shown, the discharge tubes 120, 122 are non-linear (comprise bends), which thereby aids in collecting droplets greater than 4 microns in size. Referring to FIGS. 14-20, portions of the wheeled cart 134 have been removed for ease of viewing internal components. As understood, the wheeled cart 134 may comprise the dry mist generating apparatus 100, a first liquid holding tank 144, a second liquid holding tank 146, and a waste tank 148. As best seen in FIG. 15, the liquid holding tanks 144, 146 are elevated in relation to the housing 102. In other words, the bases of the liquid holding tanks 144, 146 are higher than the base 112 of the housing 102. As a result, liquid may be gravity-fed from the one or more liquid holding tanks 144, 146 to the housing 102 (which may be positioned on a riser 101 to achieve a desired height), such as through one or more pipes 150. One or more electric valves may be used to control the flow of liquid, as discussed later herein.

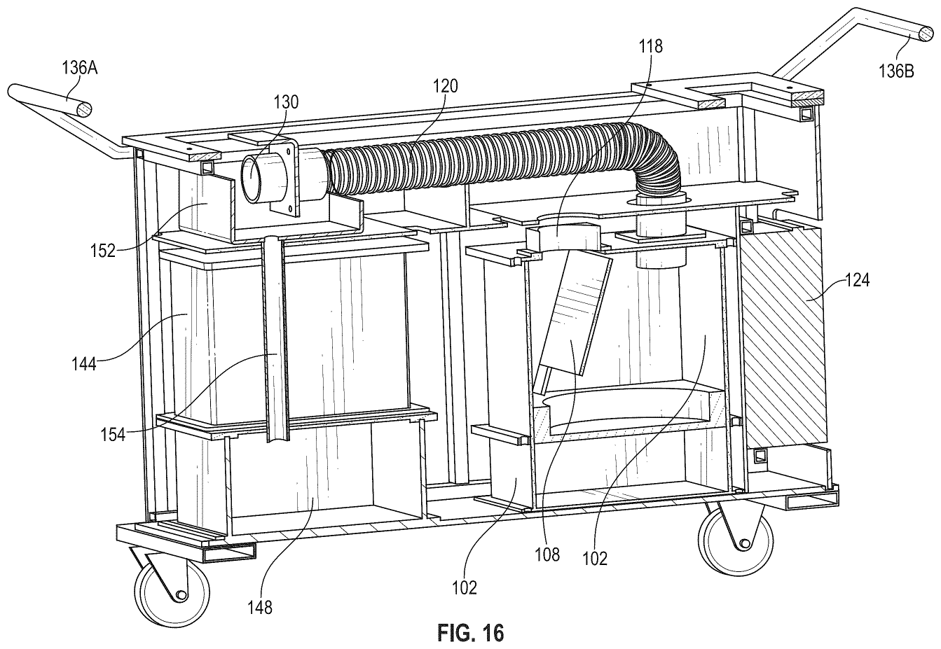

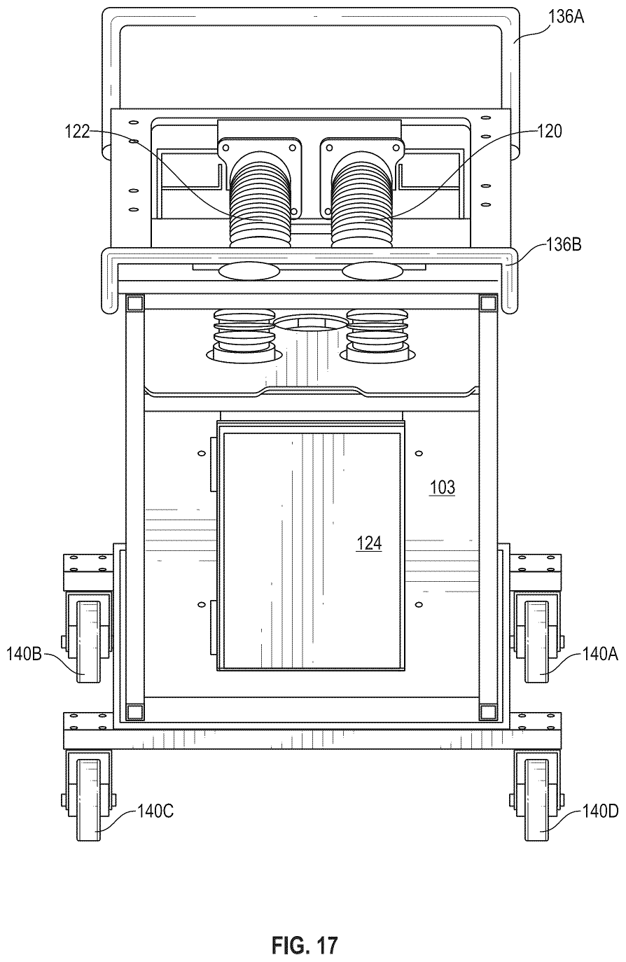

FIG. 16 illustrates a longitudinal cross-section of the wheeled cart 134 and the components therein. When closed, the exit 130, 132 of each discharge tube 120, 122 rests in a basin 152 that collects excess moisture, where it is directed to the waste tank 148 via a conduit 154. FIGS. 17-19 illustrate various angles of the wheeled cart 134 and its components.

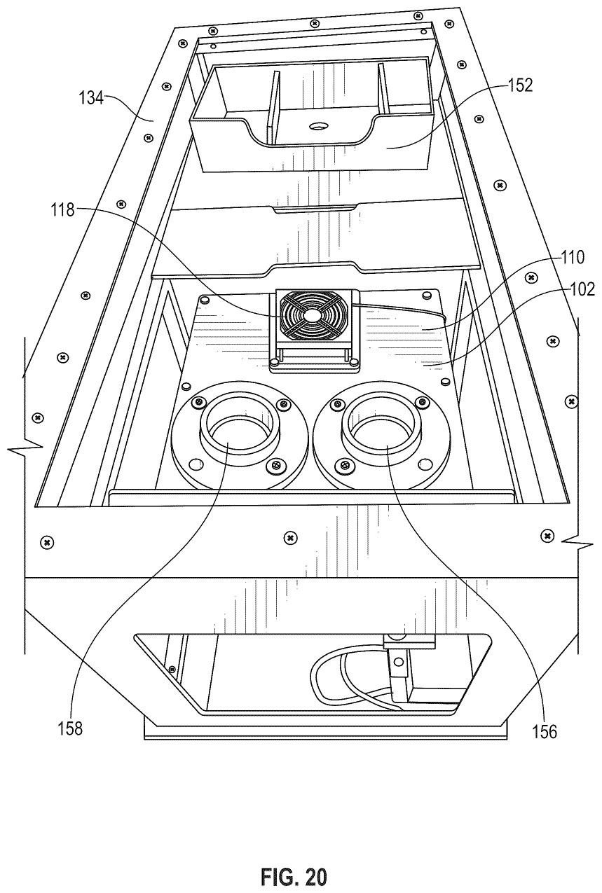

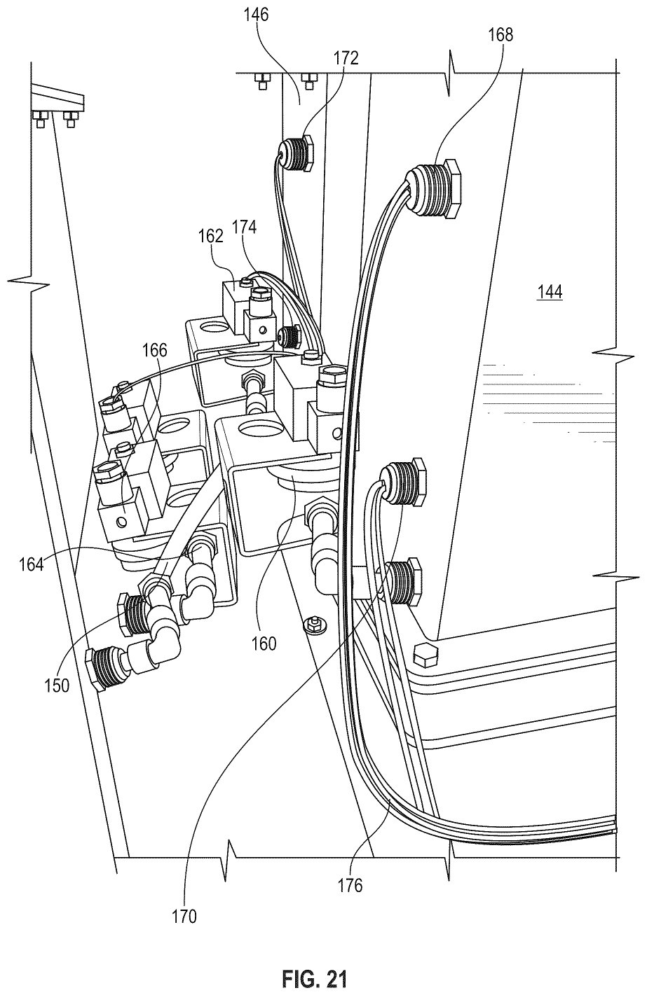

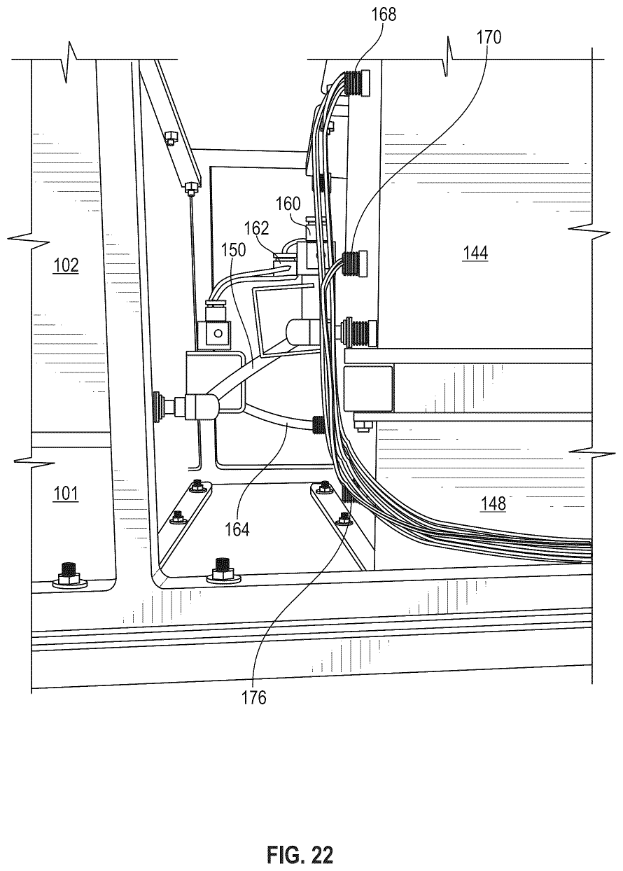

FIG. 20 illustrates a top, detailed view of the wheeled cart 134 with the hinged lid 138 removed therefrom. The discharge tubes 120, 122 have also been removed from the ports 156, 158 in this view. Turning to FIGS. 21-22, liquid (e.g., Hypochlorous Acid (HOCL)) is able to flow from a first liquid holding tank 144 and liquid (e.g., water) is able to flow from a second holding tank 146 via at least one pipe 150. The HOCL may be controlled via an electric valve 160 and the water may be controlled via an electric valve 162. It will be appreciated that a coupler may be used to combine the liquid from both tanks 144, 146 once released from their respective electric valves 160, 162 and into a single pipe 150 for conveying to the housing 102. Further, the housing may comprise an outlet pipe 164 for releasing liquid from within the housing 102 when the dry mist generating apparatus 100 is not in operation. The outlet pipe 164 may be controlled via an electronic valve 166. The outlet pipe 164 exits at a position higher than where it enters the waste tank 148 so that it may be gravity fed as well.

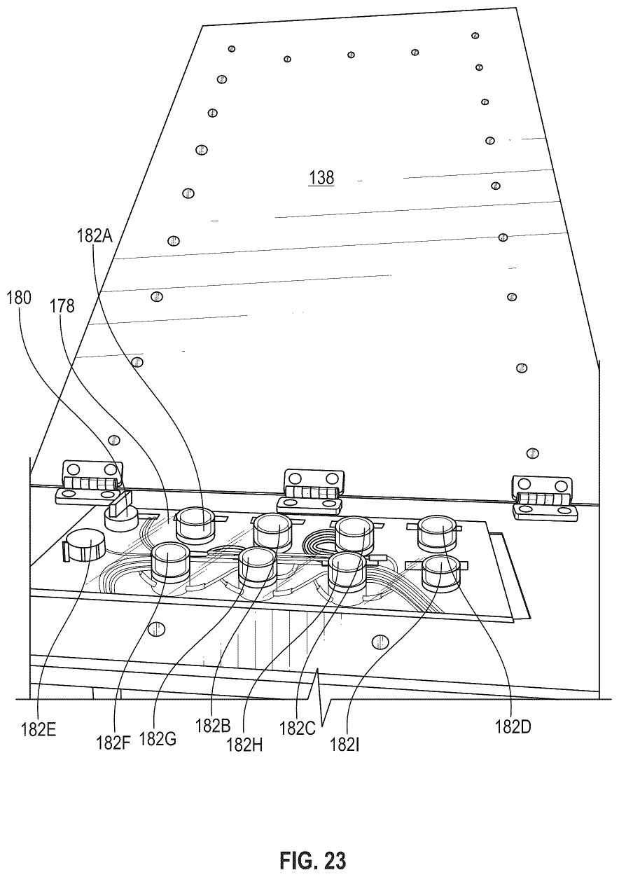

In addition, the level of liquid within each tank 144, 146, and waste tank 148 may be monitored using sensors and the controller 124. For example, the HOCL tank may comprise a “full” liquid sensor 168 and a “low” liquid sensor 170. Likewise, the water tank may comprise a “full” liquid sensor 172 and a “low” liquid sensor 174. The waste tank 148 may comprise a “full” liquid sensor 176. Each liquid sensor is capable of detecting when liquid is in contact therewith, which is read by the controller 124, which is configured to control the on/off status of the apparatus as well as provide alerts to a user. For example, referring to FIG. 23, a user control panel 178 may comprise an on/off switch 180 and a plurality of indicators and/or switches 182A-I. The indicators/switches 182A-I may alert a user to high levels of liquid, low levels of liquid, a fault or error, or may reset the controller 124. The indicators may be lights (e.g., LEDs) or audible.

In one method of use, a user would maneuver the wheeled cart 134 to the desired location for disinfecting (or humidifying, or other use), would ensure that the first liquid holding tank 144 and second liquid holding tank 146 are full of the desired liquid. This may be determined by a user by reviewing the control panel 178 and the various indicators/switches 182A-I. The controller 124 is configured to control the indicators/switches 182A-I and may thereby indicate full or low status. With both tanks 144, 146 full, a user may open the hinged lid 138 and secure it in position. With the hinged lid 138 raised, the discharge tubes 120, 122 are angled upwardly (best seen in FIGS. 12-13).

A user may then start the dry mist generating apparatus 100 using the on/off switch 180. The controller 124 then actuates the electric valves 160, 162, allowing liquid from both tanks 144, 146 to be gravity fed to the housing 102. For sanitization, the liquid may be HOCL in the first tank 144 and water in the second tank 146. The ultrasonic transducer 115 converts the liquid into droplets sized 4 microns or less (the dry mist). As the transducer 115 creates the dry mist, the fan 118 induces air into the first chamber 104 where it passes to the second chamber 106 via the air aperture 116, then upwardly to the discharge tubes 120, 122. As the air travels upward, the dry mist is likewise pulled with the air and up through the discharge tubes 120, 122. Due to the bends, angle, and length and diameter of the discharge tubes 120, 122, only dry mist (e.g., droplets of 4 microns or less) exit the discharge tubes at the exits 130, 132. Accordingly, it will be appreciated that a user may vary the angle, length, and diameter of the tubes to control the micron size of the droplets at the exits 130, 132.

The dry mist generating apparatus 100 will continue to operate until switched off by a user or until the controller 124 determines that a condition is met, such as that the run timer has reached the entered set point (10-240 minutes), or that the first tank 144 is low, the second tank 146 is low, or that the waste tank 148 is full, among other conditions. Because the HOCL exits as a dry mist, it is able to penetrate all areas of a room or building, thereby disinfecting both the air and surfaces. Because the HOCL is a dry mist, no residue remains and surfaces do not become wet—there is no need for any cleanup, which overcomes issues in the prior art.

It will be appreciated that the housing 102 may vary in size. In some embodiments, the housing 102 is sized so as to allow a user to easily carry the dry mist generating apparatus 100 for use and may comprises handles for easier carrying. In other embodiments, the housing 102 is too large to carry and must be pushed or otherwise transported using wheels either directly coupled to the housing 102 or on a cart as shown and described earlier.

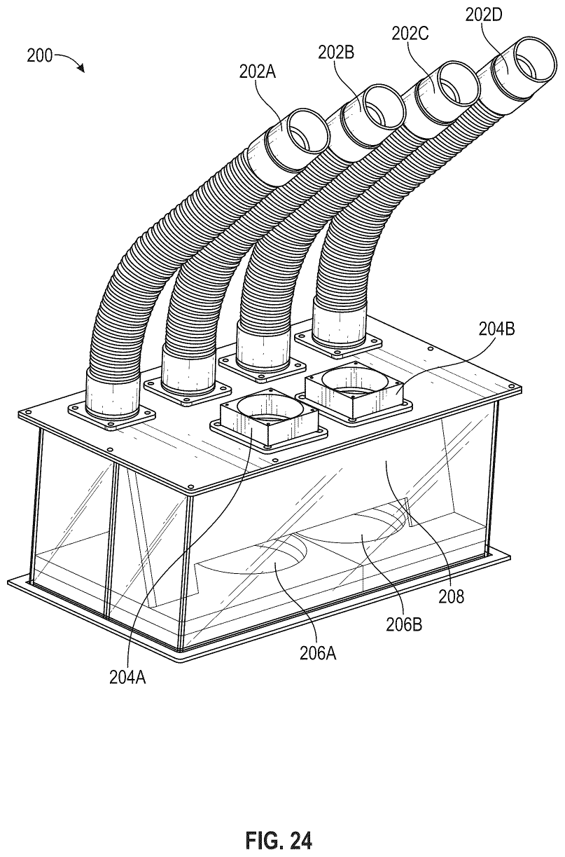

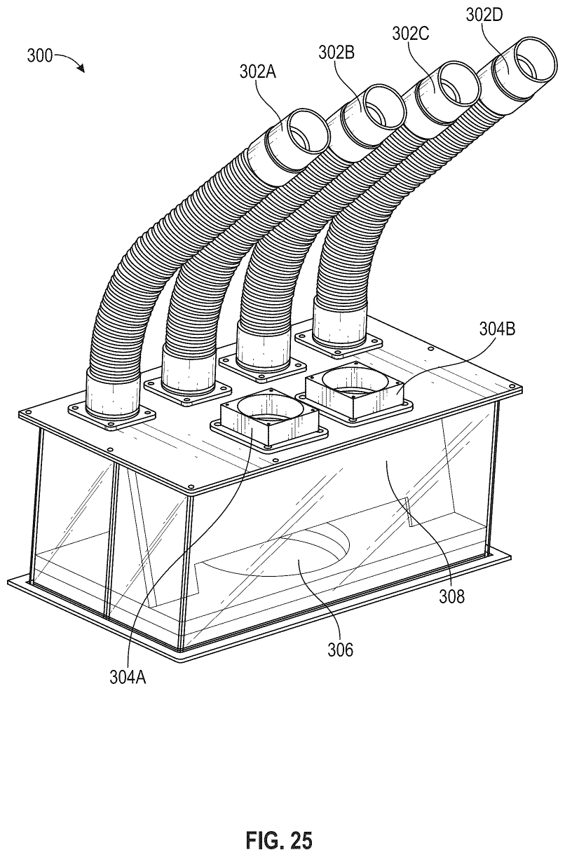

As shown in FIG. 24, the dry mist generating apparatus 200 may comprise a plurality of discharge tubes 202A-D, a plurality of intake fans 204A-B, a plurality of ultrasonic transducers 206A-B, and a diffuser plate 208. Referring to FIG. 25, a dry mist generating apparatus 300 may comprise a plurality of discharge tubes 302A-D, a plurality of intake fans 304A-B, an ultrasonic transducer 306, and a diffuser plate 308. Accordingly, it is appreciated that the present invention is not limited to the number of fans, transducers, or discharge tubes.

Further, while liquid holding tanks 144, 146 were discussed above as feeding liquid to the housing 102, other configurations may be used without departing herefrom. For example, the housing 102 may comprise one or more threaded inlets (or other couplers) allowing for a bottle or other container to be threaded thereto. One or more bottles may then feed water and/or HOCL into the housing 102, either by manual flow valves or electronic flow valves. This allows the overall size to be reduced, allowing the system to carried by a user in some embodiments. For example, the housing 102 may comprise a handle allowing a user to carry it by hand. A user may then feed the desired liquid into the housing 102 via the coupled bottles where it can be turned into dry mist. The riser 101 may also function as a waste container in such a scenario. In the alternative, a waste container may be coupled to the housing 102. While embodiments discussed herein have generally discussed a portable dry mist generating apparatus, the present disclosure is not so limited. For example, the dry mist generating apparatus 100 may be coupled to an HVAC unit or may otherwise be secured to a building. Accordingly, it will be appreciated that the dry mist generating apparatus 100 may vary in size and may be portable or a fixture. Additionally, while plastic is used as an example herein, it will be appreciated that other corrosion resistant materials may be used, such as fiberglass, aluminum, carbon fiber, and others.

Because the droplets expelled from the discharge tubes 120, 122 have, ideally, a micron size of 2 or less, the droplets are easily suspended in the air for extended times (i.e., a dry mist). However, while 2 microns or less is the preferred droplet size, it will be appreciated that any dry mist droplet (i.e., up to 9 microns) may be used without departing herefrom. This dry mist may be distributed in office spaces, schools, buildings, or any other enclosed area in need of disinfecting. Additionally, the dry mist may be distributed directly onto surfaces without creating wet surfaces. As a result, surface pathogens are neutralized. This is a significant improvement over the prior art, which does not remain airborne and causes wet surfaces.

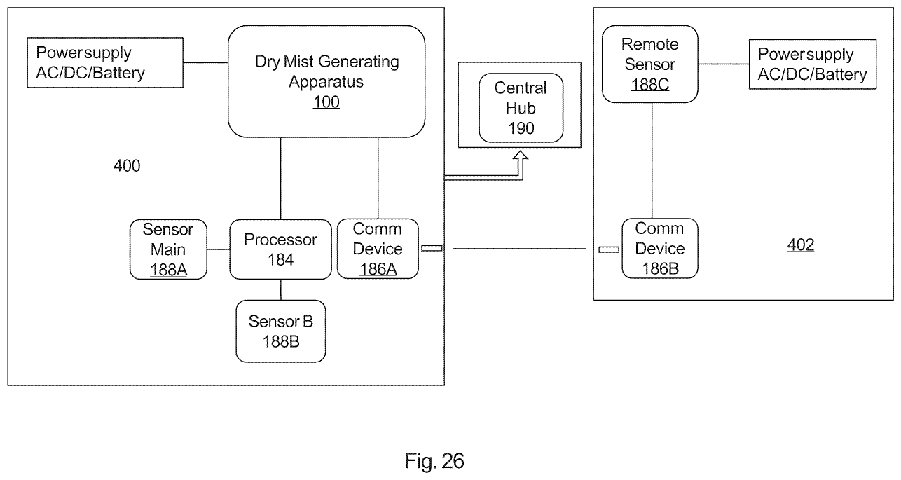

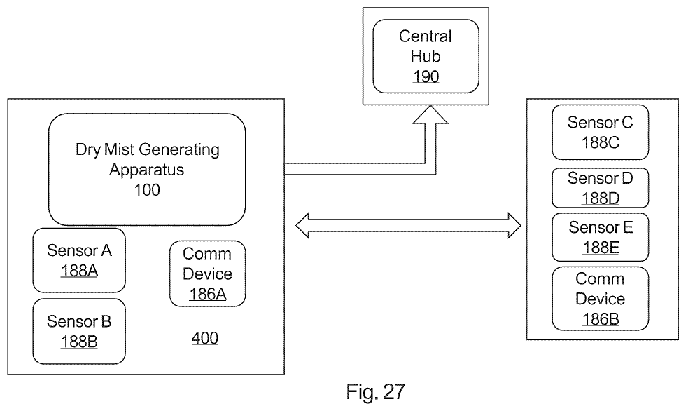

In some embodiments, as shown in FIGS. 26-27, a dry mist generating apparatus may be network-connected and configured to interface with one or more sensors, communication devices, controllers, or other components, collectively referred to as a dry mist system 400. For example, as shown in FIG. 26, the dry mist system 400 may comprise a dry mist generating apparatus 100 that is coupled to a processor 184 (e.g., CPU, microcontroller, or other processor) and a communication device 186A (e.g., wireless transceiver). The processor 184 may communicate with one or more sensors 188A-B configured to monitor the air quality of a given space. For example, when the dry mist system 400 is powered on, the one or more sensors 188A-B begin collecting data regarding the air quality, which may include ambient temperature, relative humidity, VOC levels, microbial size of particles in the air, the amount of HOCL in the air, etc. If the air quality is determined to meet a predetermined set of criteria (e.g., airborne pathogens detected), the processor 184 initiates the dry mist generating apparatus 100. The one or more sensors 188A-B continue to monitor the air until a predetermined threshold of air quality is met or exceeded (e.g., pathogens neutralized). In some embodiments, the dry mist generating apparatus 100 may, in the alternative, run for a predetermined amount of time.

In some embodiments, the dry mist system 400 may also be configured to transmit a notification to a user at a triggering event. For example, a triggering event may include sensor readings that caused the dry mist generating apparatus 100 to initiate, and may also include the status of the dry mist generating apparatus, runtime, liquid levels, etc. Data from the sensors 188A-B and the runtime and other metrics of the dry mist generating apparatus 100 may be logged and stored locally, or more preferably, in offsite cloud storage.

As shown, the dry mist system 400 may further comprise one or more remote sensor platforms 402. The remote sensor platform 402 may be positioned distally (e.g., not physically connected, such as placed across a room) to the dry mist generating apparatus 100, which may be across the same room or in a different room (a “zone”). The remote sensor platform 402 comprises one or more sensors 188C and a communication device 186B (e.g., wireless transceiver). The communication device 186B is configured to transmit data from the sensor 188C to the dry mist system 400. If the remote sensor 188C detects air quality of predetermined criteria (e.g., pathogens present), the processor 184 may initiate the dry mist generating apparatus 100. If the remote sensor 188C is in a different room or at too great a distance for the dry mist generating apparatus 100 to have an effect on the air quality, a notification may be sent to a user (either via communication device 186A or 186B), informing the user of the need to place and initiate a dry mist generating apparatus 100.

The sensors 188A-C may be configured to detect microbial sizes from 1-10 pM, which detects airborne pathogens, viruses, bacteria, and mold, among others. In some embodiments, the data from sensors 188A-C may be transmitted to an offsite or cloud-based processor (a “central hub” 190), which may be configured to monitor a plurality of sensors and zones, and to control one or more dry mist generating apparatuses 100 based on the received data. In some embodiments, the central hub 190 may be programmed to turn on/off a respective dry mist generating apparatus 100, determine how long it should run, whether to pulse (i.e., run-pause-run), or other actions. The central hub 190 may also log all events, which may be outputted to reports for review by a user, and may also notify a user, such as via push notification, cellular notification, email, etc., when a triggering event has occurred.

FIG. 27 illustrates a plurality of remote sensors 188C-E in communication with the dry mist system 400 via one or more communication device 186A-B. The processor 184 may control the dry mist generating apparatus 100 in response to received data from the one or more local sensors 188A-B and the remote (i.e., distally located) sensors 188C-E. As described earlier, data from the dry mist system 400 may be stored in the cloud (i.e., offsite data storage accessible via a network), such as in the central hub 190. A plurality of remote sensors 188C-E may be used to define “zones” or areas being monitored. The sensors 188C-E may be placed so as to overlap with one another as well, effectively monitoring the entire space. Additionally, by utilizing multiple sensors 188C-E, it may be possible to identify the direction and location of the source of contamination. Multiple zones, which may each include sensors 188C-E and/or a dry mist generating apparatus 100 (which may be coupled to local sensors 188A-B), ensure that the entire monitored space can be effectively sanitized, whether a small room, large room, multiple rooms, or an entire building. As a result, the present disclosure overcomes shortcomings of the prior art.

In one method of use, the dry mist system 400 is initiated by powering the unit on (e.g., toggle switch). Power may be received from batteries or grid power. Once the dry mist system 400 is powered on, communication is established between the various components (e.g., communication devices 186A-B, processor 184, sensors 188A-E, etc.). The processor 184 then processes data from the various sensors 188A-E to determine the current state of the dry mist generating apparatus 100 and components (e.g., fluid levels, flow, operating status, etc.), as well as the room air quality to establish a baseline reading. Based on the processed data, the processor 184 may signal to initiate the dry mist generating apparatus 100 to begin disinfection. Data may be stored locally or in the central hub 190. User alerts may be sent and reports generated from the processed and stored data. The sensors 188A-E are configured to continuously report their readings to the processor, with the dry mist generating apparatus 100 being configured to run or to cease running in response to the processed readings from the sensors 188A-E, which may be any number of triggering events, such as, but not limited to, liquid levels in the holding tank 144, liquid levels in the waste tank 148, ambient readings (e.g., temperature, humidity, microbial concentration, HOCL concentration, etc.).

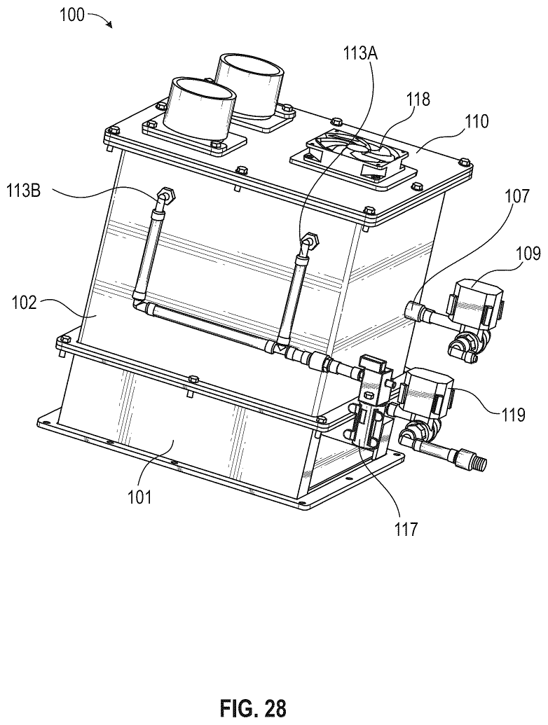

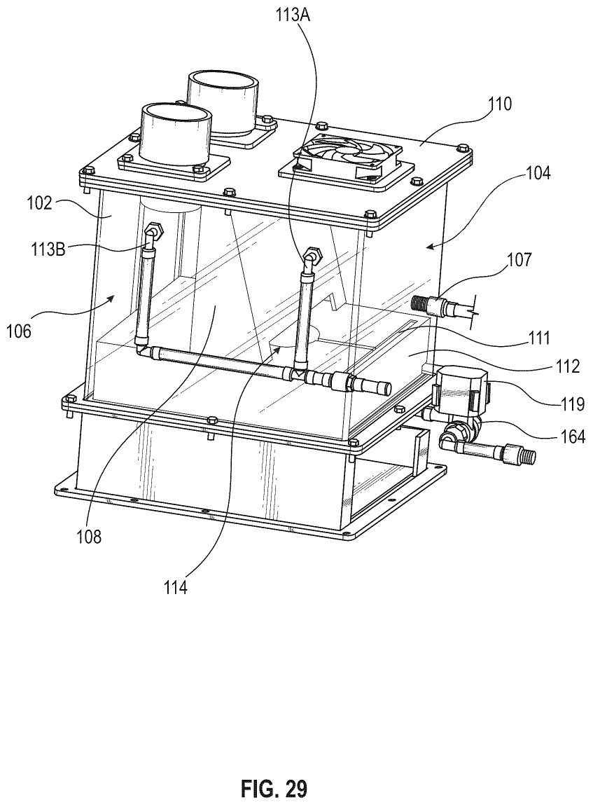

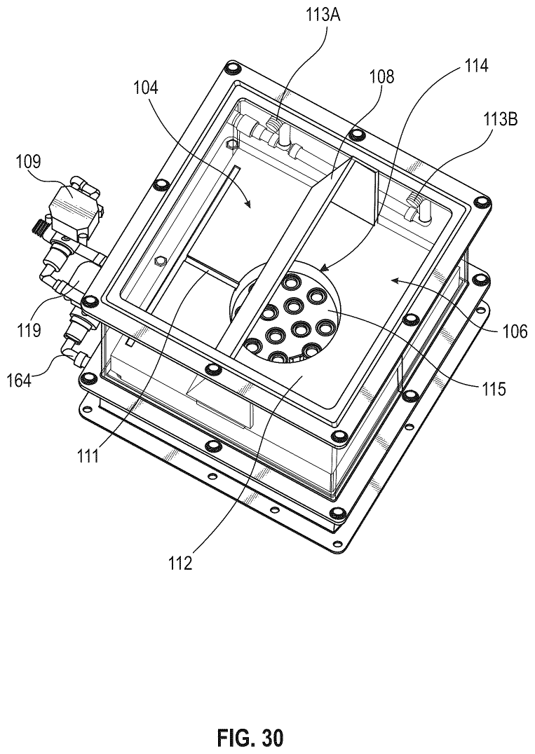

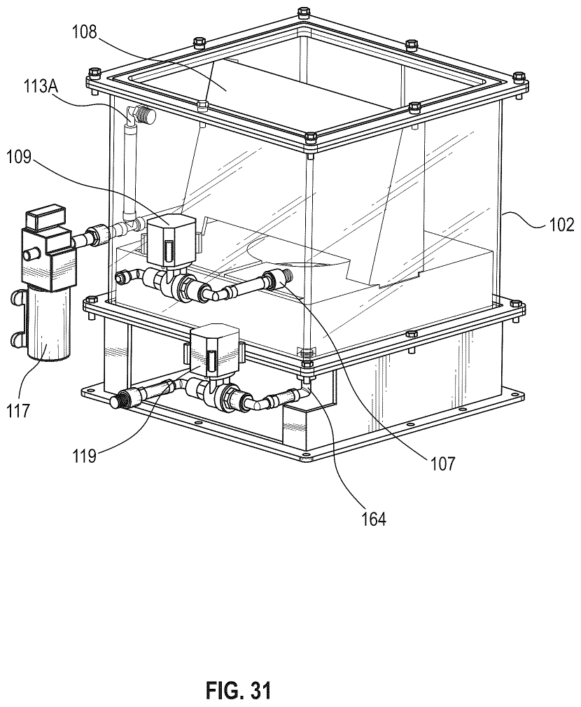

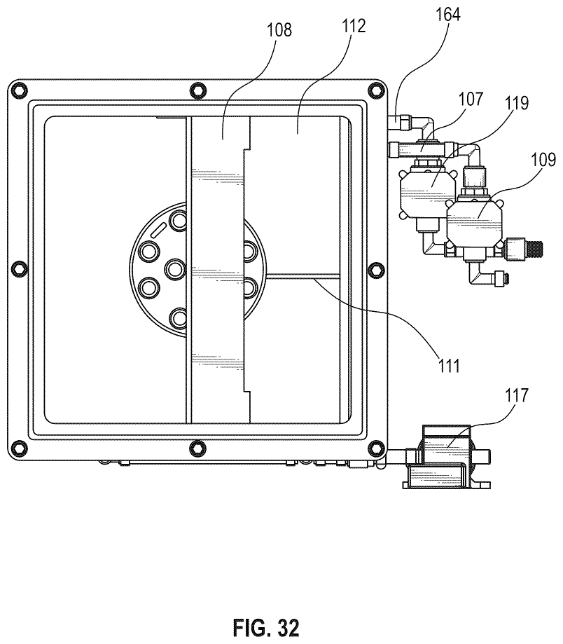

It will be appreciated that the configuration of the dry mist generating apparatus 100 may vary in configuration without departing herefrom. For example, FIGS. 28-35 illustrate embodiments of a dry mist generating apparatus 100 and wheeled cart 134 therefor. More particularly, FIGS. 28-32, illustrate that the dry mist generating apparatus 100 may comprise a first inlet 107 that may be gravity fed from the first liquid holding tank 144, which may contain HOCL. A ball valve 109 (or other valve/solenoid) may be used to control the flow of liquid from the liquid holding tank 144 to the first chamber 104 above the base 112. As a result, the HOCL may easily flow into the ultrasonic transducer aperture 114. The base 112 may comprise one or more channels 111 to aid in guiding the flow of liquid into the ultrasonic transducer aperture 114.

A second inlet 113A may be positioned near the top 110 of the first chamber 104, with a third inlet 113B positioned near the top 110 of the second chamber 106. The second inlet 113A and third inlet 113B are coupled to a pump 117 to force liquid (e.g., water) into the first and second chambers 104, 106, respectively. The second inlet 113A and third inlet 113B ideally comprise spray heads configured to fully rinse the first and second chambers 104, 106, respectively, with pressurized water (or other liquid) via the pump 117.

The outlet pipe 164 may be positioned on the underside of the dry mist generating apparatus 100, allowing waste liquid to gravity feed into the waste tank 148. A second ball valve 119 (or other valve/solenoid) may control the flow of waste liquid into the waste tank 148.

The ultrasonic transducer aperture 114 may also be of various diameters and depths. A smaller diameter allows for less liquid to be required to operate, which allows for shorter run times and higher efficiency (i.e., less HOCL required). The one or more channels 111 help direct the liquid on the base 112 into the ultrasonic transducer aperture 114 and to the ultrasonic transducer 115.

FIG. 33 illustrates a perspective view of the flow restricting plate 121, as discussed earlier herein. FIG. 34 illustrates a front elevation view of the flow restricting plate 121.

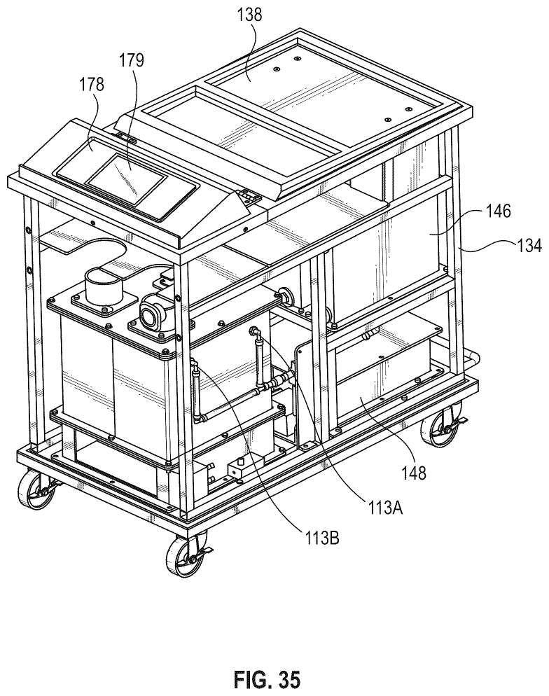

Referring to FIG. 35, the wheeled cart 134 comprises a control panel 178. In some embodiments, as shown, the control panel 178 may comprise a touchscreen 179 to facilitate both input and output. The touchscreen 179 is connected to a computing device to facilitate the processing and display of output in addition to receiving and processing user input. The computing device may be programmed to allow multiple users to individually access and control the dry mist generating apparatus 100, custom programming of run times or actions to take based upon triggering events, remote control access, GPS tracking, operation logs, programming and operational updates, among other features. The computing device may comprise components known in the art, such as memory, processors (e.g., CPU, controller, microcontroller), transceivers, sensors, etc., capable of achieving the above-mentioned features. Wireless transceivers facilitate communication with remote sensors (e.g., sensors 188C-E), with remote control devices, with network devices for being internet-connected (such as to communicate with central hub 190), smartphones, tablets, laptops, or other wireless devices. Such wireless capabilities allows a user to control and monitor the status of the dry mist generating apparatus 100 from a distance.

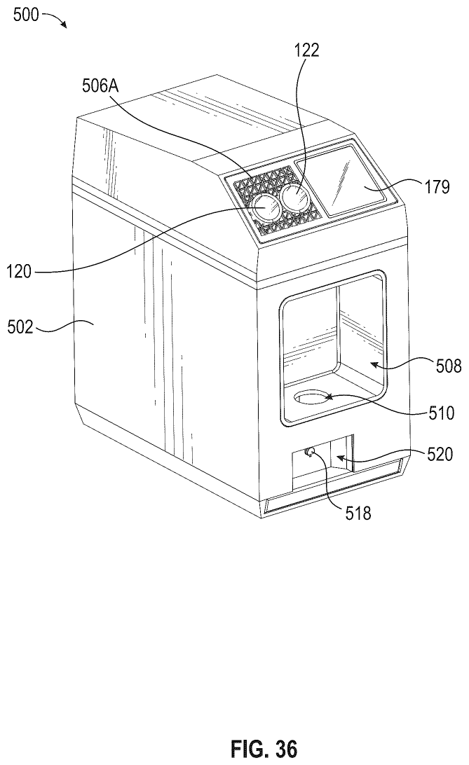

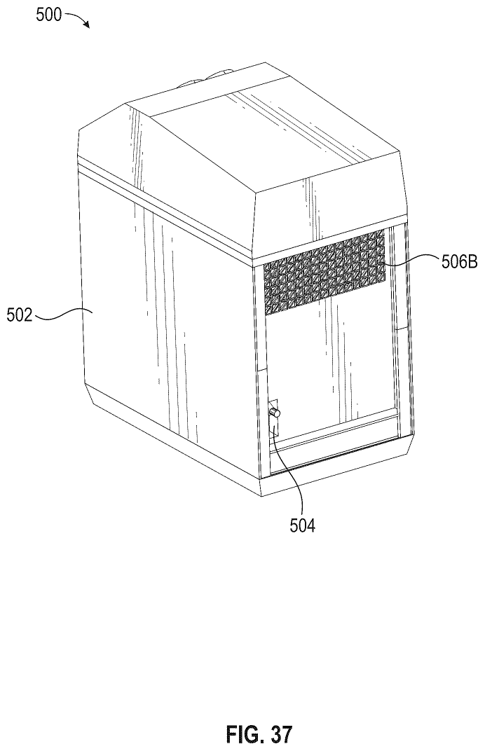

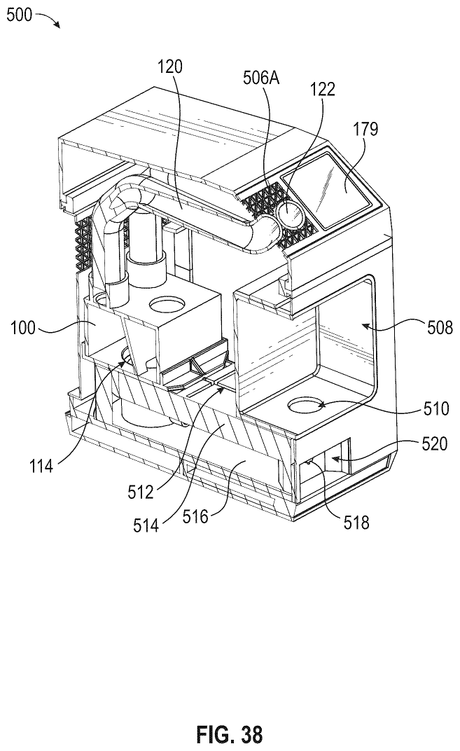

While the dry mist apparatus 100 has been shown and described as a standalone device and as part of a wheeled cart 134, it will be appreciated that other housings, containers, or formfactors may be used as part of a system. For example, referring now to FIGS. 36-38, a dry mist system 500 comprises an enclosure 502, the enclosure 502 containing the dry mist apparatus 100 therein (seen in the cross-section of FIG. 38). Similar to the wheeled cart 134, the dry mist system 500 may comprise a touchscreen 179 for user input and output. The one or more discharge tubes 120, 122 may exit the enclosure 502 in a variety of positions, such as at the front top next to the touchscreen 179. As appreciated, this configuration is optimal as it provides for the bends in the discharge tubes 120, 122 to facilitate droplets of larger sizes condensing and falling back into the second chamber 106.

The enclosure 502 may comprise one or more power connectors 504, which may facilitate providing power (e.g., grid power, generator, battery, etc.) to the dry mist system 500. The enclosure 502 may further comprise one or more ventilation screens 506A-B to allow for heat dissipation within the enclosure 502. In some embodiments, the enclosure 502 is preferably sized so as to be portable, allowing a user to place the enclosure 502 on a counter, table, desk, etc. Receptacle 508 therefore provides a convenient mechanism to refill the unit with hypochlorous. For example, a swappable reservoir (e.g., a bottle; not shown) may be placed with the reservoir opening in the receptacle opening 510, allowing the swappable reservoir to feed hypochlorous, via hydrostatics, to the ultrasonic transducer 115 in the dry mist generating apparatus 100.

As shown, the hypochlorous may flow through one or more channels 512 in center member 514 and into the ultrasonic transducer aperture 114. In other words, the one or more channels 512 couple the receptacle opening 510 to the ultrasonic transducer aperture 114 to direct the flow to the ultrasonic transducer aperture 114. Excess liquid or waste liquid may accumulate in waste tray 516, which is positioned beneath the center member 514. A user may empty the excess liquid via the spigot (or other valve) 518, which may be located in a recess 520, although not required.

Accordingly, a user may easily add a swappable reservoir (e.g., a bottle) of hypochlorous to the dry mist system 500 when desired. In other words, when the hypochlorous within a first swappable reservoir is emptied, a user may remove the swappable reservoir and replace it with a second swappable reservoir. In some instances, a user may simply refill an empty swappable reservoir and replace it. The user may interact with the screen 179 to thereby control the dry mist system 500 as described earlier herein. Additionally, the dry mist system 500 may likewise comprise sensors (as described earlier herein), which may be located within the enclosure 502, distally thereto (e.g., across the room), or both.

While described above as using hydrostatics, it will be appreciated that motors and pumps may also be used without departing herefrom.

Accordingly, the dry mist generating apparatus 100, 200, 300 and dry mist systems 400 and 500 solve the problems in the art, namely, the need for a disinfectant that can remain airborne for a significant amount of time, that may penetrate small spaces, that does not leave surfaces wet, that may produce this dry mist in an apparatus that is not susceptible to corrosion and other component failures, that operate independently based on one or more sensor readings, and that may be remotely controlled and monitored.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A dry mist system, comprising:
a dry mist generating apparatus, comprising:
a diffuser plate positioned within a housing and extending from a top of the housing to a base of the housing, the diffuser plate positioned non-perpendicularly to the top of the housing and the base of the housing and separating the housing into a first chamber and a second chamber;
an ultrasonic transducer aperture in the base of the housing configured to receive an ultrasonic transducer, the diffuser plate comprising an air aperture straddling the ultrasonic transducer aperture;
a first inlet positioned in the first chamber and above the base of the housing, the first inlet coupled to a first liquid tank comprising hypochlorous acid, the hypochlorous acid configured to flow from the first liquid tank, through a first valve, and into the first chamber;
a second inlet positioned in the first chamber near the top of the housing, the second inlet coupled to a pump, the pump coupled to a second liquid tank comprising water;
a third inlet positioned in the second chamber near the top of the housing, the third inlet coupled to the pump, the pump coupled to the second liquid tank comprising water;
a fan positioned on the top of the housing and configured to force air downward into the first chamber, wherein the air is compressed to a first pressure into a lower portion of the first chamber, the compressed air passing through the air aperture of the diffuser plate and over the ultrasonic transducer as it passes to a bottom portion of the second chamber, the air decompressing in the bottom portion to a second pressure less than the first pressure; and
at least one discharge tube coupled to the top of the housing above the second chamber, wherein sub-10 micron-sized droplets in the second chamber flow upwardly to the at least one discharge tube.

2. The dry mist system of claim 1, wherein the base comprises one or more channels configured to direct the flow of hypochlorous acid into the ultrasonic transducer aperture.

3. The dry mist system of claim 1, wherein the at least one discharge tube comprises a flow restricting plate within a cuff of the at least one discharge tube.

4. The dry mist system of claim 1, further comprising a user control panel.

5. The dry mist system of claim 4, wherein the user control panel comprises a computing device and a touchscreen.

6. The dry mist system of claim 5, wherein the computing device comprises a processor and a wireless transceiver.

7. The dry mist system of claim 6, further comprising a plurality of sensors.

8. The dry mist system of claim 7, wherein at least one of the plurality of sensors is positioned distally to the dry mist generating apparatus and is configured to communicate to the computing device of the dry mist generating apparatus via a second transceiver wirelessly coupled to the wireless transceiver of the computing device.

9. The dry mist system of claim 8, wherein the computing device is configured to wirelessly communicate with a central hub.

10. The dry mist system of claim 8, wherein the plurality of sensors are configured to monitor:
i. ambient temperature,
ii. relative humidity,
iii. microbial concentration, and
iv. hypochlorous acid concentration.

11. The dry mist system of claim 1, further comprising a controller configured to control an on/off status of the ultrasonic transducer and the fan upon determining a status of:
a. first and second liquid sensors of the first liquid tank,
b. first and second liquid sensors of the second liquid tank, and
c. a full liquid sensor of a waste tank.

12. The dry mist system of claim 1, further comprising a wheeled cart.

13. A dry mist system, comprising:
a dry mist generating apparatus, comprising:
a diffuser plate positioned within a housing and extending from a top of the housing to a base of the housing, the diffuser plate positioned non-perpendicularly to the top of the housing and the base of the housing and separating the housing into a first chamber and a second chamber;
an ultrasonic transducer aperture in the base of the housing configured to receive an ultrasonic transducer, the diffuser plate comprising an air aperture straddling the ultrasonic transducer aperture;
a first inlet positioned in the first chamber and above the base of the housing, the first inlet coupled to a first liquid tank comprising hypochlorous acid, the hypochlorous acid configured to flow from the first liquid tank, through a first valve, and into the first chamber;
a second inlet positioned in the first chamber near the top of the housing, the second inlet coupled to a pump, the pump coupled to a second liquid tank comprising water;
a third inlet positioned in the second chamber near the top of the housing, the third inlet coupled to the pump, the pump coupled to the second liquid tank comprising water;
a fan positioned on the top of the housing and configured to force air downward into the first chamber, wherein the air is compressed to a first pressure into a lower portion of the first chamber, the compressed air passing through the air aperture of the diffuser plate and over the ultrasonic transducer as it passes to a bottom portion of the second chamber;
at least one discharge tube coupled to the top of the housing above the second chamber, wherein sub-10 micron-sized droplets in the second chamber flow upwardly to the at least one discharge tube;
an outlet pipe coupled to the waste tank, the outlet pipe comprising a valve;
a processor;
a first wireless transceiver; and
at least one sensor positioned distally to the dry mist generating apparatus, the at least one sensor coupled to a second wireless transceiver;
wherein the processor retrieves and processes data from the at least one sensor via the first and second wireless transceivers, the processor configured to determine an on/off status of the dry mist generating apparatus.

14. A dry mist system, comprising:
an enclosure;
a dry mist generating apparatus positioned within the enclosure;
one or more discharge tubes coupled to the dry mist generating apparatus and at least partially exiting the enclosure;
the enclosure comprising a receptacle configured to receive a reservoir, the receptacle comprising a receptacle opening to receive a liquid from the reservoir therein;
one or more channels coupling the receptacle opening to an ultrasonic transducer aperture; and
a waste tray for collecting waste liquid.

15. The dry mist system of claim 14, wherein the waste tray is coupled to a valve configured to drain liquid from the waste tray.

16. The dry mist system of claim 14, further comprising one or more ventilation screens in the enclosure.
